(19) United States
(12) Patent Application Publication
(10) Pub. No.: US 2008/0108012 A1
(43) Pub. Date: May 8, 2008

(54) Dental photographic tool

(76) Inventor: Jonathan MacArthur, London (CA)

Correspondence Address: VALENTINE A COTTRILL, 50 QUEEN STREET NORTH, STE. 1020, P.O. BOX 2248, KITCHENER, ON, N2H6M2, CA

(21) Appl. No.: 11/976,361

(22) Filed: Oct. 24, 2007

Related U.S. Application Data
(60) Provisional application No. 60/853,764, filed on Oct. 24, 2006.

(51) Int. Cl. A61B 1/253

(52) U.S. Cl. 433/31

(57) ABSTRACT

The present invention provides a dental photographic tool and more particularly a dental photographic tool to assist in intra-oral photography using extra-oral cameras. The dental photographic tool being portable and optionally heated and including a mirrored portion that is releasably connected to a handle portion.

This application claims the benefit of the U.S. Provisional Patent Application No. 60/853,764, filed on Oct. 24, 2006.

### FIELD OF THE INVENTION

The present invention relates to a dental photographic tool and more particularly to a dental photographic tool for use in intra-oral photography using extra-oral cameras.

### BACKGROUND OF THE INVENTION

Conventional dental photographic techniques utilize hand-held mirrors in order to photograph entire arches or quadrants/sextants of teeth. Fogging, collapse of soft tissue, i.e. cheeks or tongue, over the mirror perimeter and the intrusion of the holder's fingers within the photograph are all acknowledged problems with the current technique. In many cases 3 operators may be required to take a photograph including one to retract tissue(s), one to hold the mirror, and another to hold the camera. Since dental auxiliaries working alone are often called upon to perform this task, the man-power requirement poses an obvious problem.

Suggestions have been advanced to reduce the fogging issue including: (1) pre-heating the mirror with hot water and (2) applying a stream of air over the mirror face. Suggestion (1) is only effective up to the time that the heat dissipates at which point the mirror must be removed, re-heated and re-positioned at the suitable angle with tissue(s) retracted. Suggestion (2) often leads to spray droplets being deposited on the mirror marring the photograph. The air spray area is often insufficient to maintain the entire mirror fog-free.

Some manufacturers have applied handles to the mirrors. These handles are usually rigid, non-hinged, inflexible and not rotatable making them difficult for the patient to maintain in the correct position for ideal lighting and perspective. These handles have therefore not eliminated the man-power issue.

Tissue retractor does not seem to have been addressed and is still a major issue with these conventional mirrors and techniques.

In addition, most extra-oral cameras use a flash of some kind but achieving an illuminated field sufficient to achieve focus remains difficult as it relies on the synchronization of mirror angle and the overhead operator's light. Photographic magnification is achieved solely through the magnification capabilities of the camera and can be a limiting factor.

### SUMMARY OF THE INVENTION

The present invention provides a dental photographic tool to assist in intra-oral photography using extra-oral cameras.

In one aspect the present invention provides a dental photographic tool comprising a head portion including a mirrored portion and a flexible handle portion releasably connected to the head portion and operable to allow for rotation of the head portion about the connection therebetween. The handle portion may include a gripping portion having a flexible neck portion extending therefrom, the flexible neck portion being operable to releasably couple to the head portion.

In a further embodiment, the present invention provides a dental photographic tool comprising a handle portion including a gripping portion and a flexible neck extending therefrom and a head portion including at least one mirrored surface, the head portion operable to releasably connect to the handle portion along an axis about which the head portion is rotatable.

### BRIEF DESCRIPTION OF THE DRAWINGS

The invention will be described below in further detail with particular reference to the accompanying drawings in which:

FIG. 1 illustrates one embodiment of the dental photographic tool of the present invention;

FIG. 2 is a photograph of an alternative embodiment of the dental photographic tool showing the mirror face and a switch on the handle;

FIG. 3 is a photograph of the dental photographic tool of FIG. 2 showing the side of the tool;

FIG. 4 is a photograph of the dental photographic tool of FIG. 2 showing the neck portion in a bent configuration;

FIG. 5 is a close up photograph of the mirror portion of the dental photographic tool of FIG. 2;

FIG. 6 is a close up photograph of the handle portion of the dental photographic tool of FIG. 2; and

FIG. 7 shows an alternate embodiment of the dental photographic tool of the present invention.

### DETAILED DESCRIPTION OF THE PREFERRED EMBODIMENTS

The present invention provides a dental photographic tool for use in intra-oral photography and in particular for use in intra-oral photography using extra-oral cameras. The dental photographic tool is described by reference to the attached Figures in which the dental photographic tool is indicated generally at numeral 10.

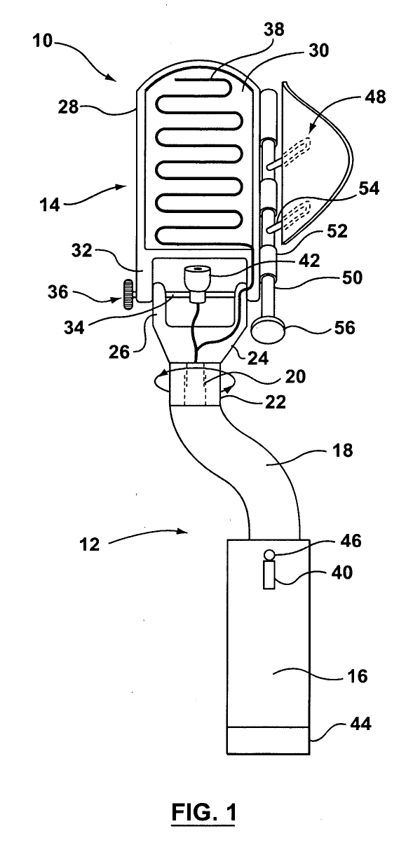

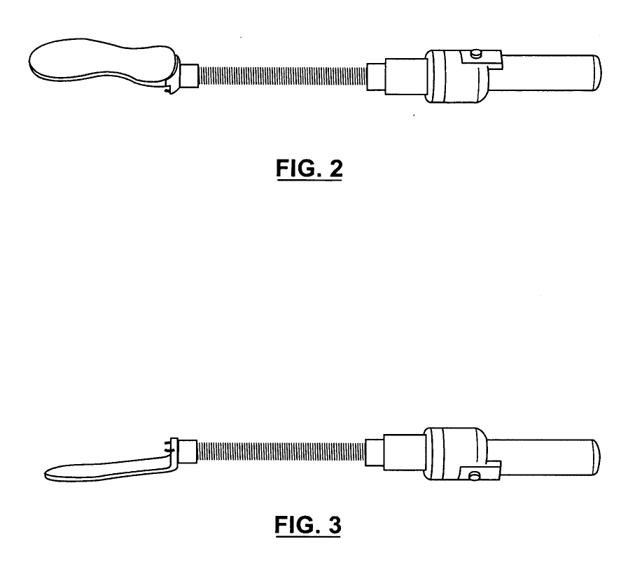

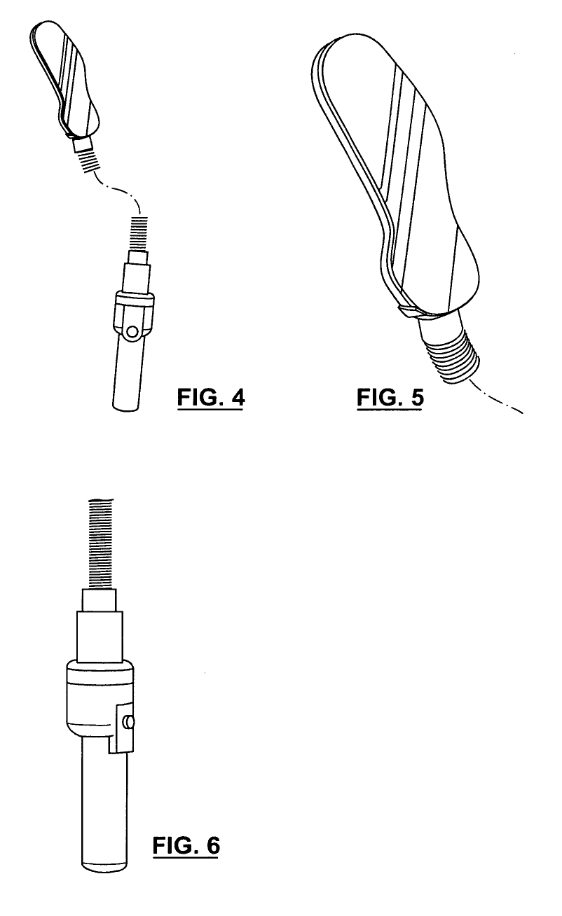

As shown in FIG. 1, the dental photographic tool 10 includes a handle portion 12 connected to a head portion 14. The handle portion 12 includes a gripping portion 16 and is connected to the handle portion 12 via a flexible neck 18.

The handle portion 12 is appropriately shaped for its function as a handle and may be cylindrically shaped as shown, or may comprise another shape that facilitates its function, for example, generally cylindrical with varying diameters along its length. The gripping portion 16 of the handle portion may include additional features for a comfortable grip including, for example, formed finger grips therein to permit a secure hold by the user. The gripping portion is preferably sized and shaped to be held by an adult hand to reinforce a secure hold by a user.

Extending from one end of the handle portion 12 is flexible neck 18. The attachment of flexible neck 18 to the handle portion 12 may be by any suitable connection means known to a person skilled in the art. At the opposite end from the handle portion 12, the flexible neck 18 is connected to the head portion 14. Flexible neck 18 extends between the handle portion 12 and the head portion 14 a sufficient distance for suitable mirror placement when in use. It will be understood that this distance may vary depending on the requirements of the end use. An example of a suitable length of the flexible neck 18 is approximately 6 to 8 inches long. However this is not meant to be limiting and merely serves as an example. In one embodiment the flexible neck is made from flexible metal covered by a plastic casing or coating suitable for contact within the mouth of a patient.

As can be seen in FIG. 1, the head portion 14 is coupled to the flexible neck 18 by a connector 20. In the illustrated embodiment, connector 20 includes an upper portion 24 and a lower portion 22 that is rotationally coupled to the flexible neck 18. The lower portion 22 is releasably attached to the neck portion 18 and is operable to rotate about the neck portion 18 to allow for rotation of the head portion 14 by a user for accurate positioning as required. The upper portion 24 of the connector 20 is connected to the head portion 14. and allows for rotation/pivoting of the head portion 14 about the connection point. In the illustrated example, the upper portion 24 includes two prongs 26 that extend from the outer edges of the upper portion 24 to connect to the head portion 14 described in further detail below.

It will be understood that the connector 20 may be releasably attached/detached from the handle portion 12 at the lower edge of the lower portion 22 or may alternatively be releasably attached/detached from the upper portion 24. The releasable attachment of the head portion 14 via connector 20 allows a user to remove the head portion 14 for sterilization or alternatively to remove the head portion 14 and replace it with an alternate head portion 14 of a different size and/or shape or magnification capability. It will therefore be understood that the head portion may be made in a variety of sizes and/or shapes that are operable to releasably connect to the handle portion described herein.

In the illustrated embodiment in FIG. 1, the head portion 14 includes frame 28 containing mirror 30. It will be understood that the frame 28 may be a separate component from mirror 30, made from materials sufficient to support the mirror therein and suitable for insertion into a patient's mouth. Suitable materials include plastic, coated metals and the like. Alternatively, the frame 28 may be made from material that includes a mirrored surface.

The mirrored surface may cover the entire head portion 14 or may cover only one surface of the head portion or alternatively may cover distinct portions of the head portion. At one end, the frame 28 includes spaced leg portions 32 that extend from the frame 28. These leg portions 32 are operable to connect to the prongs 26 on the connector 20. The connection of these two parts allows for rotation of the head portion 14 about the axis of the connection. As can be seen in FIG. 1, in the illustrated embodiment the connection is made using a pin 34 that extends through the leg portions 32 and the prongs 26 approximately perpendicular to the flexible neck 18. The head portion 14 is operable to rotate about the axis of the pin 34 and a user may also secure the head portion 14 in a fixed position by tightening knob 36 located at one end of the pin 34. Other connections may be used to connect the head portion 14 to the connector 20 that may optionally provide rotational movement of the head portion 14 about the connection point. It will also be understood that the head portion 14 may be connected directly to the flexible neck 18 in a similar manner, and that the head portion 14 may connected to either connector 20 or neck 18 in a manner that does not permit rotation as described.

As stated above, the head portion 14 includes a mirror 30. The mirror 30 may be located on one or both surfaces of the head portion 14. The mirror may be any shape or size depending on the user's requirements. For example, the illustrated embodiment includes a mirror that is substantially rectangular in shape having rounded corners. The mirror may alternatively have a rounded shape including circular or oval in shape, or comprising a partially rounded shape, e.g. semi-circular or semi-oval. One embodiment may include a 1:1 mirrored surface (non-magnified) on one side and a magnified mirrored surface on the other.

In one embodiment as illustrated, the head portion 14 includes heating means 38 for heating the mirror 30 to minimize or prevent fogging of the mirror. The illustrated heating means 38 comprises grid wire(s) that are attached, for example using glue, to the back of the mirror 30. In the case of a two-faced mirror, the wire(s) may be sandwiched between the two faces. The wire(s) are connected to a power supply 44 via a switch 40 within the handle portion 12 and may be run through the flexible neck 18 or between the neck 18 and an outer coating on the neck, if applicable, to control activation of the heating of the wire(s). The wire(s) are operable to heat the mirror within 1-2 minutes after the switch 40 is depressed. In order to permit detaching of the head portion from the handle, the wire is adapted for disconnection at connector 20 incorporating, for example, a plug assembly.

The photographic tool 10 of the present invention may also include a light 42. In the illustrated embodiment, the light 42 is mounted on the pin 34 and is operable to supply light to the area being photographed. The light 42 may be mounted in order to be operable to rotate about the pin 34. Alternatively, the light 42 may be mounted in a non-rotatable fashion, for example, on connector 20. The light 42 may connected to the same power supply being used for the heating means 38, if present, or may be connected to a separate power supply activated by a separate switch. Preferably the light 42 is a light emitting diode (LED).

The power supply/battery 44 of the photographic tool 10 is located in the handle portion 12. The power supply 44 may be in the form of a removable battery that is operable to supply sufficient power to the heating means 38 and/or the light 42, for example a 4.8 v 1.65 AL NiMH rechargeable battery, or the equivalent in non-rechargeable batteries. The switch 40 that supplies the power may also include indicating means 46, such as a light or an audible sound, for indicating when the battery requires recharging. In an alternative embodiment, the photographic tool 10 may include a power supply 44 that is not removable but includes a connection point for attachment to a recharging unit.

The photographic tool of the present invention may also include at least one retractor plate 48. The retractor plate 48 is rotatably mounted on at least one side of the head portion 14. The retractor plate 48 may vary in size and shape depending on the specific needs of the photographic situation. In the illustrated embodiment, the retractor plate 48 is connected to the head portion 14 using retractor pin 50. The retractor pin 50 is connected to the head portion 14 along one edge thereof being received in a series of housing tubes 52 formed in the edge of the head portion 14. Extending from the retractor pin 50 are protrusions 54 that extend from the pin 50 between the housing tubes 52. These protrusions 54 are connected to the retractor plate 48 and allow for rotation of the retractor plate 48 about the axis of the pin 50. In the illustrated embodiment, the housing tubes 52 include 3 separate 5-8 mm diameter tubes. The tubes 52 are separated from each other by approximately 2 cm breaks. However, it will be understood that the photographic tool 10 is not limited to this particular construction and may include tubes of varying size provided that they are operable to receive the retractor pin 50 therethrough and allow for rotation of the pin 50 about their axis. Further, it will be understood that the attachment of the retractor plate 48 is not limited to the illustrated example. The retractor plate 48 may be connected to the head portion 14 by any means known to a person skilled in the art that allows for rotation of the retractor plate 48 about the connection point and further that allows for the retractor plate 48 to be releasably connected to the head portion 14 so that it may be removed for cleaning and/or sterilisation if required.

The photographic tool 10 may further comprise more than one retractor plate 48. The photographic tool may comprise a plurality of retractor plates 48 releasably connected to the head portion 14 and operable to rotate about their connection point when in use.

When the position of the retractor plate 48 is obtained whether it be 90° or 120° from the plane of the mirror (either up or down), the retractor pin 50 may be pushed forward, i.e. toward the mouth, and be locked in place. As an example, the pin 50 may include a tapered frictional fit at both ends which provides frictional resistance to rotation. Alternatively, the pin 50 may include octagonal ends that may be positioned into an octagonal female receptacle which prevents further rotation of the pin 50 and therefore the retractor plate 48. Further, a combination of both approaches may be used. Alternatively, other means to lock the rotation of the pin 50 may be used that will be known to a person skilled in the art. As an example, the desired angle of the retractor plate 48 may be adjusted by rotating a dial 56 located at the outermost extension of the pin 50 outside the mouth. This dial 56 may then be pushed forward into the tapered fitting and/or octagonal locking chamber. In order to remove the photographic tool 10 from the mouth the retractor plate 48 may be released from a locked position and folded flat unto the mirrored surface of the head portion 14.

The retractor plate 48 may be made of white reflective material or may be mirrored. The retractor plate 48 serves to hold tissue in the mouth, for example the cheek and/or the tongue, away from the area being photographed. Additionally, the retractor plate 48 serves to reflect light towards the mirror providing additional light for a photograph. When not in use, the retractor plate 48 may be removed or locked against the mirror face protecting it from damage while not in use or during sterilization. Where a tool in accordance with the invention is adapted for use with a plurality of retractor plates 48, it will be understood that a user may only require the use of one of the plurality of plates 48 and may, therefore, lock or remove the additional plates 48 during use.

The mirror 30 of the head portion 14 may also include a magnified portion, or alternatively may be a magnified mirror version.

When sterilisation of the photographic tool 10 is required the retractor plate 48 and the light 42 may be removed from the tool 10. The head portion 14 may then be disconnected from the handle portion 12 at the connector 20 and may be placed in a sterilization bath. The retractor plate 48 may also be sterilized. It will be understood that the parts that require sterilization will be made from material that can withstand continuous sterilization.

The remaining parts of the photographic tool 10 are not removed for sterilization in a sterilization bath may be made from a material that can withstand cleaning using disinfectant solutions, for example, disinfectant wipes.

In the embodiment which includes a mirror 30 on one side of the head portion 14 it will be understood that the opposite surface of the head portion 14 will be made from or include a non-heat conducting material to prevent heat transference to tissue(s).

In an alternative embodiment, the photographic tool may include at least one lip shield, not illustrated, as well as or in place of a retractor plate 48.

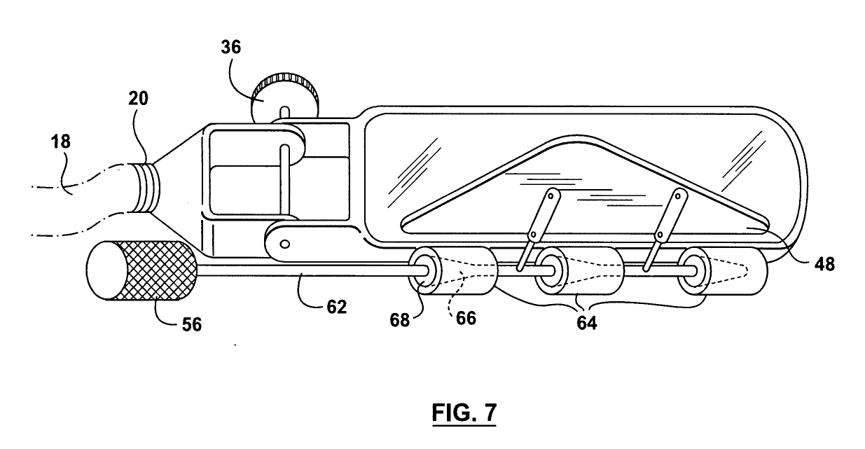

In an alternate embodiment, shown in FIG. 7, the tissue retractor 48 on the photographic tool 10 is connected to the mirror frame 28 using a shaft 62 that connects through a series of tapered hinges 64. Each of tapered hinges 64 are connected to the head portion 14 of the photographic tool. The tapered hinges 64 each include a channel 66 for receiving shaft 62. Located on one side of each of these tapered hinges 64 is a tapered opening 68 leading into the channel 66. The terminal hinge 64 located adjacent the top of the head portion 14 includes a tapered opening 68 and at least a partial channel 66, as shown. The shaft 62 includes at least three tapered portions that correspond to the shape of the tapered openings 68 on the hinges 64. In assembly, shaft 62 is positioned to be threaded through the channels 66 so that each of the tapered portions on the shaft 62 are located in a position to be received in and out of the tapered openings 68. The shaft 62 is operable to be rotated and/or pulled away from the hinges 64, thereby releasing the tapered portions from the tapered openings 68 or rotated towards/pushed towards the hinges 64 thereby frictionally engaging the tapered portions within the tapered openings 68. This movement and securement of the shaft allows a user to rotate the retractor 48 that is connected to the shaft 62, as discussed above in relation to pin 50, and secure the retractor 48 in a desired location for use. Located at the end of shaft 62 away from the hinges 64 is position knob 56 that provides a user with an easy gripping portion for manipulating the shaft 62.

In use, the dental photographic tool 10 is placed in the patient's mouth at a suitable position for a photograph to be taken. In order to place the photographic tool in the appropriate position, the user may bend the flexible neck 18 and/or rotate the head portion 14 about the pin 34 and/or rotate the connector 20 or a combination of any of the above. If the user requires the use of at least one retractor plate 48, then the retractor plate 48 is connected to the head portion 14 prior to placing the tool 10 in the patient's mouth. The retractor plate 48 is initially folded onto the mirrored surface until a portion of the tool 10 including at least a portion of the mirrored surface is positioned adjacent or inside the patient's mouth and then the retractor plate 48 is positioned to hold back the tongue and/or tissue by rotating the dial 56 on the retractor pin 50. The heating means 38 and/or light 42 may be activated by the user using the switch 40 located on the handle portion 12 about 1 to 2 minutes prior to placement in the mouth.

While this invention has been described with reference to illustrative embodiments and examples, the description is not intended to be construed in a limiting sense. Thus, various modifications of the embodiments described herein, as well as other embodiments of the invention, will be apparent to persons skilled in the art upon reference to this description. It is therefore contemplated that the appended claims will cover any such modifications or embodiments. Further, all of the claims are hereby incorporated by reference into the description of the preferred embodiments.

1. A dental photographic tool comprising:
a head portion comprising a mirror; and
a handle portion releasably connected to the head portion, said head portion being rotatably mounted onto the handle portion.

2. The dental photographic tool according to claim 1, comprising a flexible neck portion extending between the handle portion and the head portion, the flexible neck portion being releasably coupled to the head portion.

3. The dental photographic tool according to claim 1, wherein the head portion includes a frame and a mirror received within the frame.

4. The dental photographic tool according to claim 1, wherein the head portion is made from reflective material.

5. The dental photographic tool according to claim 1, further comprising heating means within the head portion positioned to heat the mirror.

6. The dental photographic tool according to claim 5, further comprising a power supply connected to the heating means for supplying power thereto.

7. The dental photographic tool according to claim 5, wherein the heating means comprises at least one heating wire.

8. The dental photographic tool according to claim 1, further comprising at least one retractor plate releasably and rotatably mounted on the head portion.

9. The dental photographic tool according to claim 1, further comprising at least one light mounted adjacent to the mirror.

10. The dental photographic tool according to claim 9, wherein the light is releasably mounted thereon and operable to rotate about the mounted position.

11. The dental photographic tool according to claim 9, wherein the light is connected to a power supply.

12. The dental photographic tool according to claim 1, wherein the head portion is attached to the handle portion with a connector that is operable to disconnect from at least one of the head portion and the handle portion.

13. The dental photographic tool according to claim 6, comprising at least one switch located in the handle portion and operable to activate the power supply.

14. A dental photographic tool comprising:
a handle portion comprising a gripping portion and a flexible neck extending therefrom; and
a head portion comprising at least one mirrored surface, the head portion operable to releasably connect to the handle portion along an axis about which the head portion is rotatable.

15. The dental photographic tool according to claim 14, further comprising at least one retractor plate releasably attached to the head portion.

16. The dental photographic tool according to claim 14, further comprising at least one light source releasably connected thereto.

17. The dental photographic tool according to claim 14, further comprising heating means operable to heat the at least one mirrored surface.

18. The dental photographic tool according to claim 17, wherein the heating means comprises at least one heating wire located on the underside of the mirrored surface.

19. The dental photographic tool according to claim 14, wherein the head portion is substantially flat and comprises two opposing surfaces each including a mirrored portion.

20. The dental photographic tool according to claim 14, wherein the head portion comprises at least one mirrored surface and at least one insulated surface on the underside of the mirrored surface.
